(19) United States
(12) Patent Application Publication
(10) Pub. No.: US 2026/0157761 A1
(43) Pub. Date: Jun. 11, 2026

(54) SURGICAL HANDPIECE AND COOPERATING ROTARY TOOL

(71) Applicant: EMD KFT., Debrecen (HU)

(72) Inventor: Mihály Gyula UJVÁRI, Debrecen (HU)

(73) Assignee: EMD KFT., Debrecen (HU)

(21) Appl. No.: 18/708,392

(22) PCT Filed: Nov. 11, 2022
(86) PCT No.: PCT/HU2022/050081

§ 371 Date: May 8, 2024

(30) Foreign Application Priority Data
Nov. 12, 2021 (HU) P2100390

(51) Int. Cl. A61B 17/16

(52) U.S. Cl. CPC A61B 17/1631; A61B 17/1622

(57) ABSTRACT

The present invention relates to a surgical handpiece (100) comprising a housing (102) and a drive shaft (10) arranged therein, wherein a stem-engaging bore (11) is formed in the drive shaft (10), which is open at a distal end (10a) of the drive shaft (10) and has a longitudinal axis (t) common to the drive shaft (10), at least three flanged bores (12) are formed in the wall of the drive shaft (10) along its circumference perpendicular to the longitudinal axis (t), opening into the stem-engaging bore (11) , whose flange (12a) is on the inner side of the wall of the drive shaft (10) facing the stem-engaging bore (11) , a fixing ball (13) is fitted in each of the flanged bores (12), and at the outer side of the wall of the drive shaft (10) a ball retainer (20) is provided which is movable between closed and open positions, said ball retainer (20) is arranged in the closed position to wedge the fixing balls (13) into the flanged bore (12) and in the open position to allow the partial exit of the fixing balls (13) from the flanged bore (12). The invention also relates to

The invention relates to a surgical handpiece comprising a housing and a drive shaft arranged therein, wherein a stem-engaging bore is formed in the drive shaft, which is open at a distal end of the drive shaft and has a common longitudinal axis with the drive shaft.

The invention further relates to a rotary tool adapted to be received in a surgical handpiece according to the invention, comprising a shaft having a longitudinal axis and a surgical head. The surgical head may be of any configuration, such as a drill head, a milling head, a ball milling head, a diamond milling head, a twist drill head, etc.

### BACKGROUND ART

In many cases, surgical handpieces are used for drilling, milling or other surgical operations that are performed with a rotary tool clamped into the surgical handpiece. Therefore, a surgical handpiece typically has a motor-driven drive shaft that transmits rotary motion to the rotary tool that is clamped into the handpiece by transmitting torque. The torque is typically transmitted by a form-locking connection between the distal end of the drive shaft and the proximal end of the rotary tool, while the rotary tool is clamped independently. An example of such a surgical handpiece is the perforator shown in patent document WO2015/150844 A1, where the torque is transmitted through a coupling profile at the distal end of the drive shaft and a complementary coupling profile at the proximal end of the drill bit, while the drill bit is secured by a pin passing through a through-hole of the drill bit perpendicular to the longitudinal axis of the drive shaft.

This type of mounting does not allow for quick and easy replacement of the rotary tool, and in addition, it requires complex machining of the rotary tool to create the correct mounting profile.

It is an objective of the invention to provide a surgical handpiece and a rotary tool that can be clamped therein, and which are free from the drawbacks of state-of-the-art solutions. It is an objective of the present invention to provide a surgical handpiece and a rotary tool can be clamped therein, wherein the mechanism for clamping the rotating stem also provides for the torque transmission and thus for the rotation of the rotary tool.

It is a further objective of the invention to provide a rotary tool that can be clamped in such a surgical handpiece.

The above objectives are achieved by a surgical handpiece according to claim 1.

The invention further relates to a rotary tool according to claim 16.

Preferred embodiments of the invention are defined in the dependent claims.

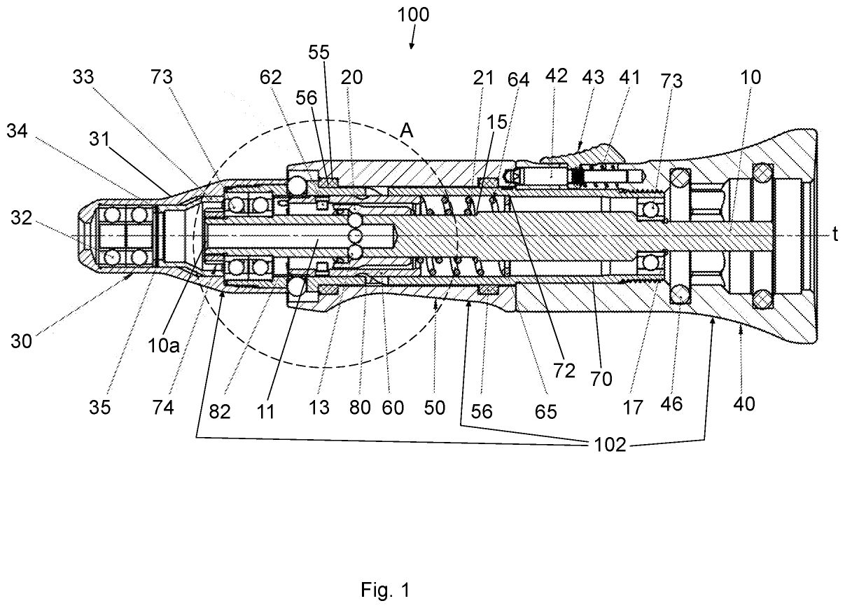

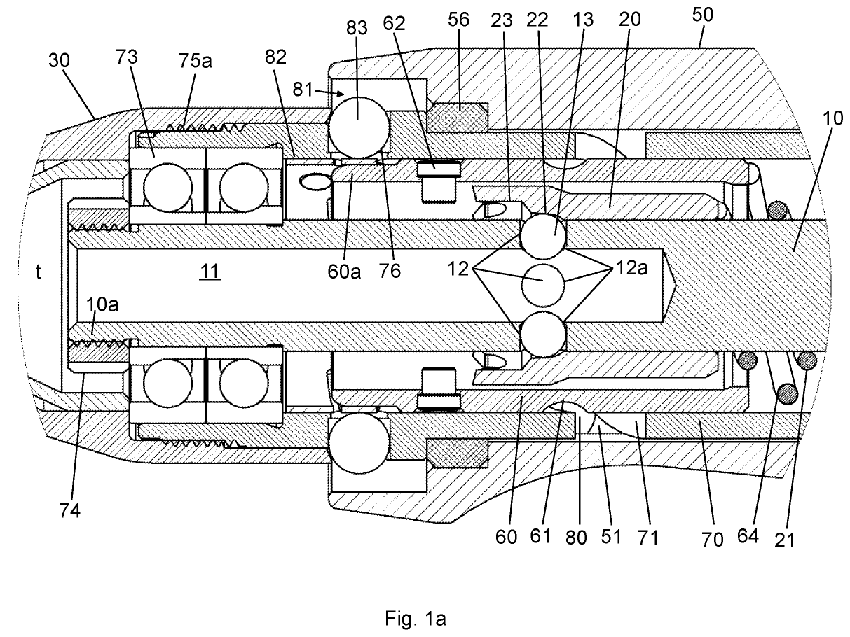

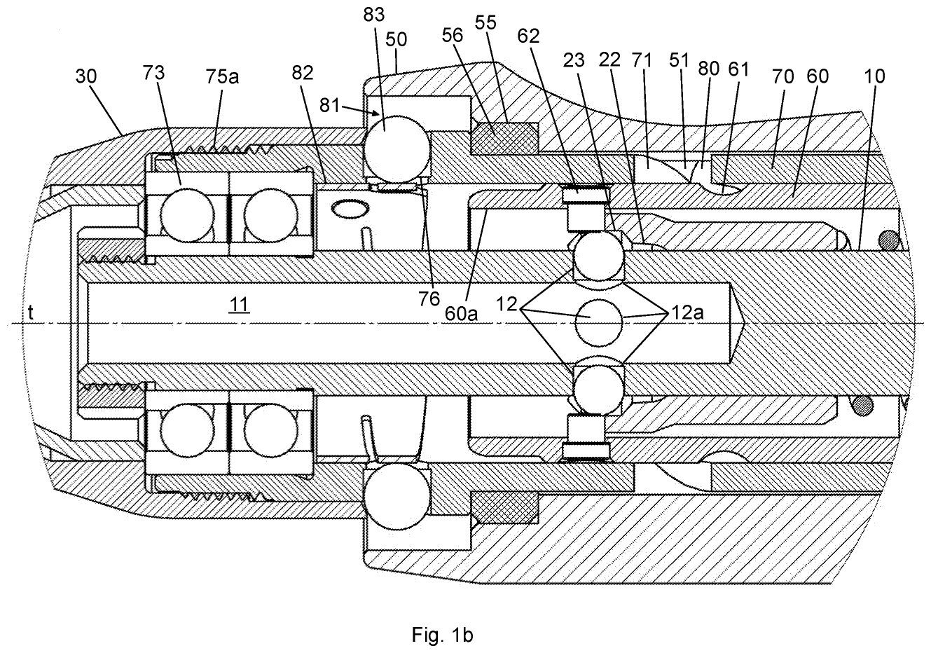

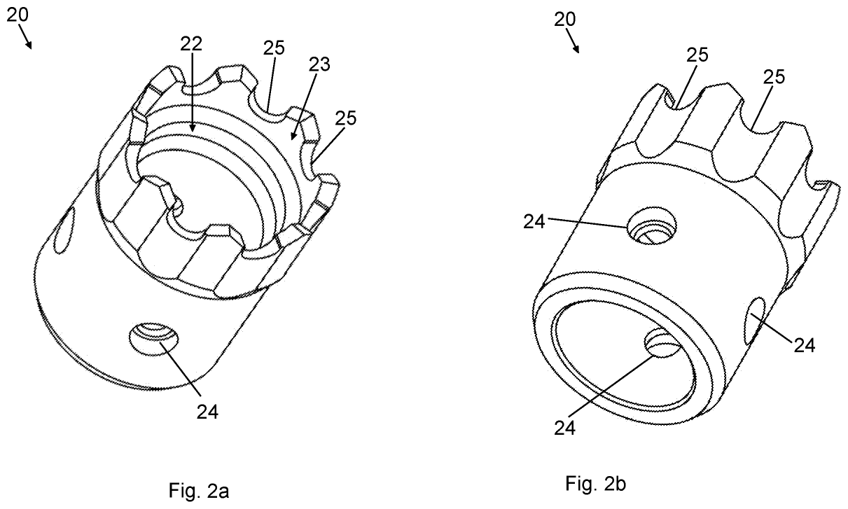

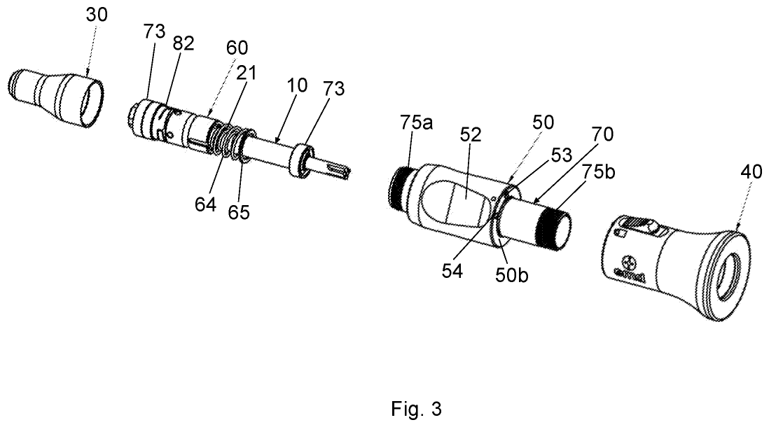

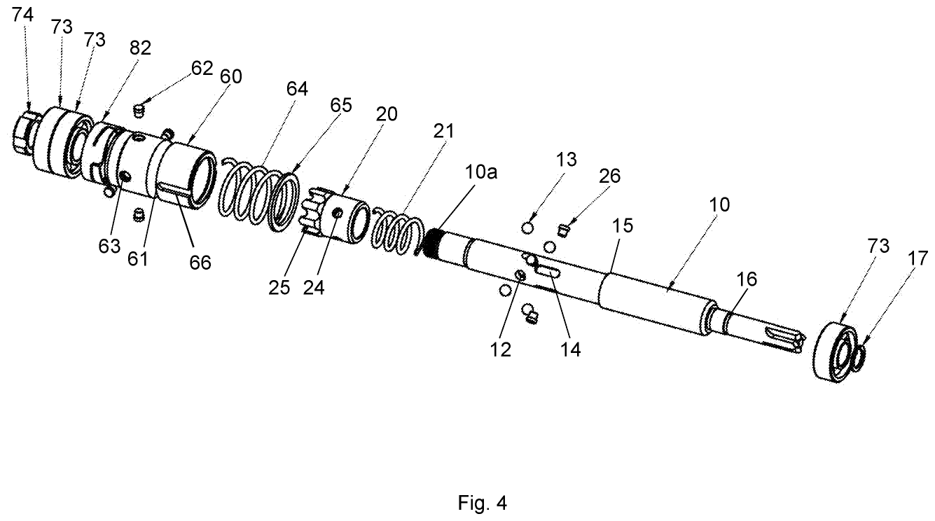

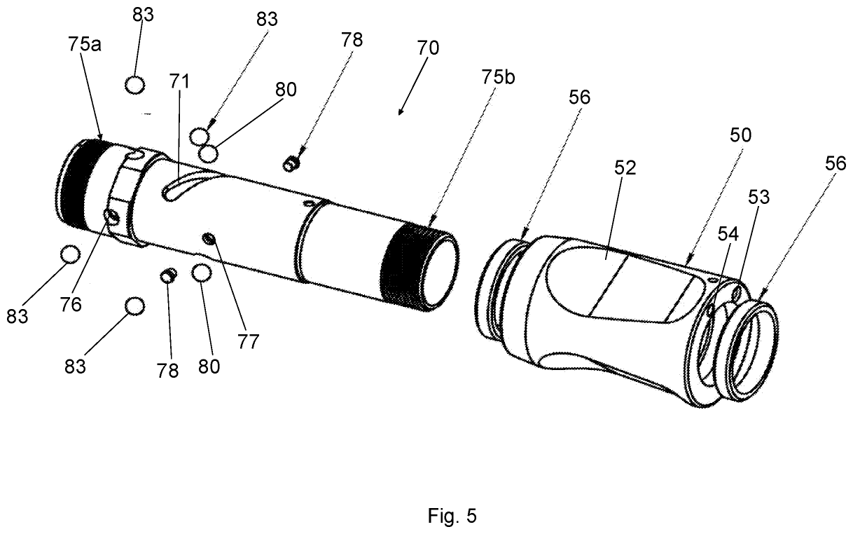

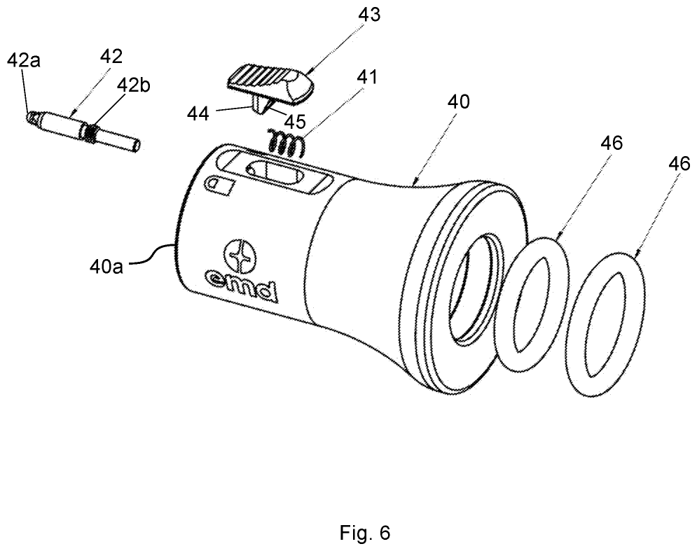

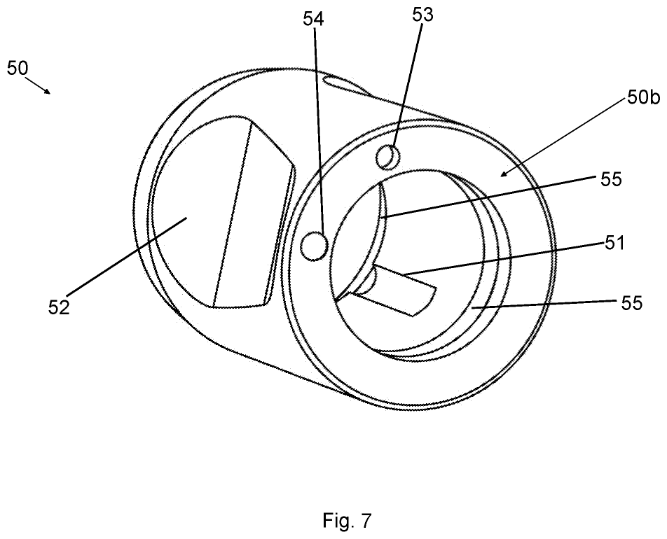

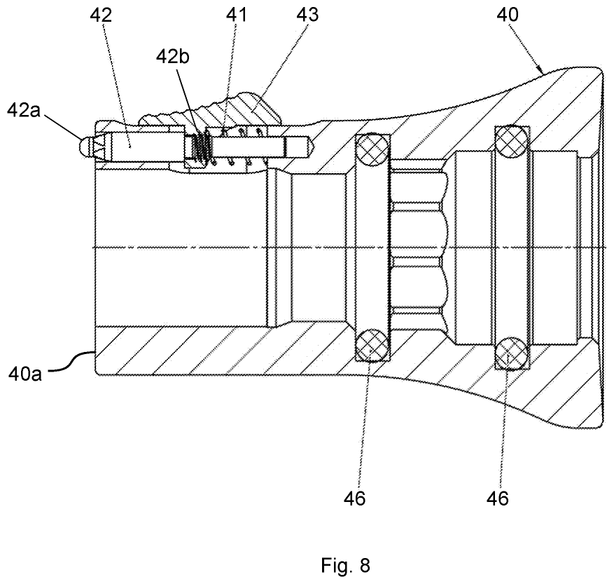

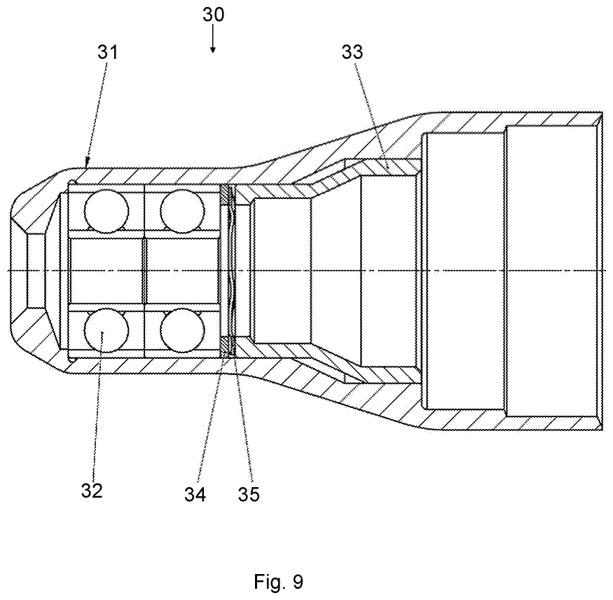

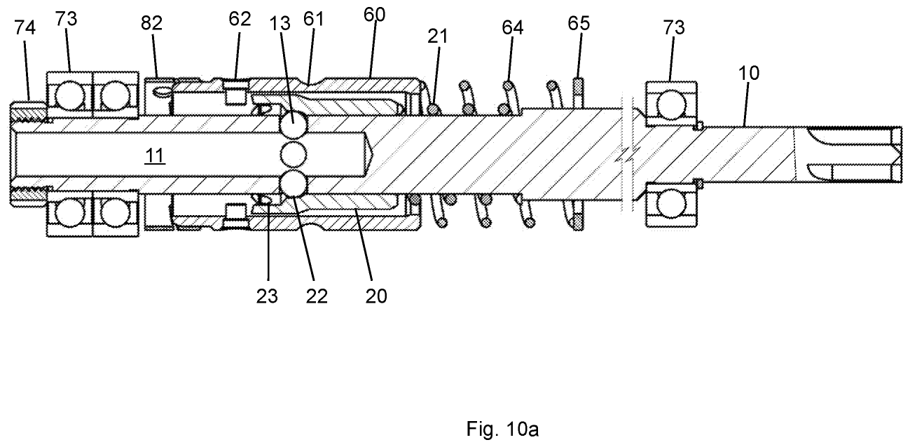

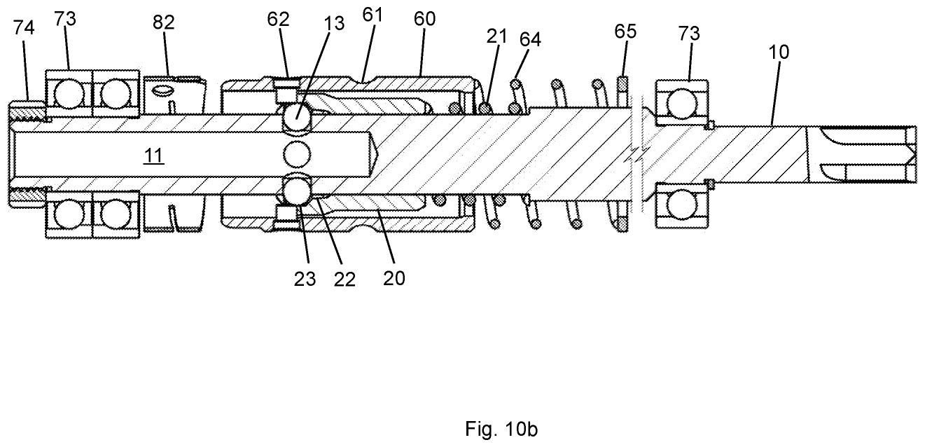

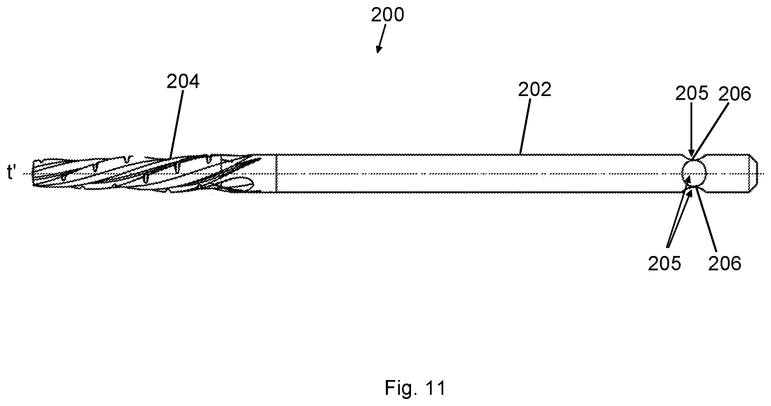

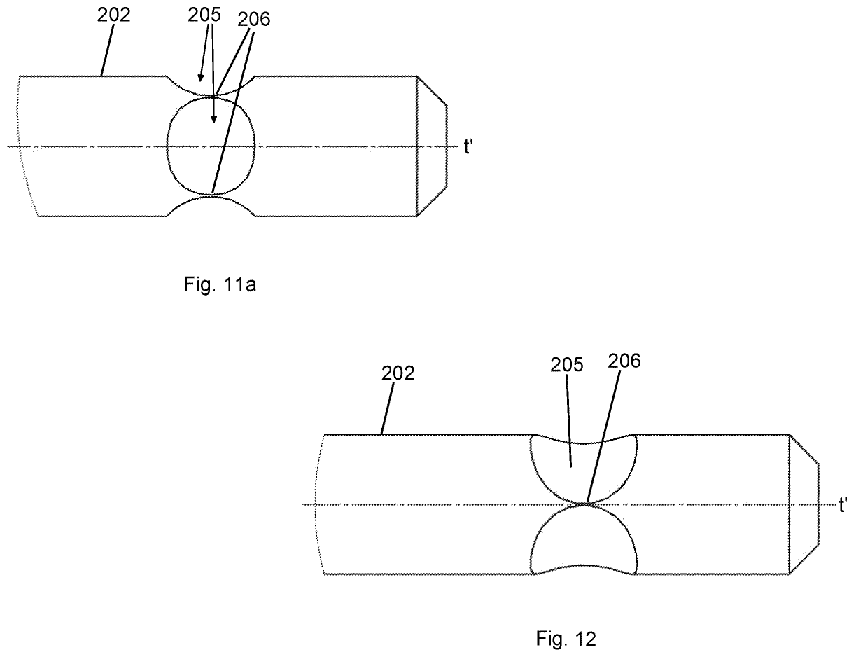

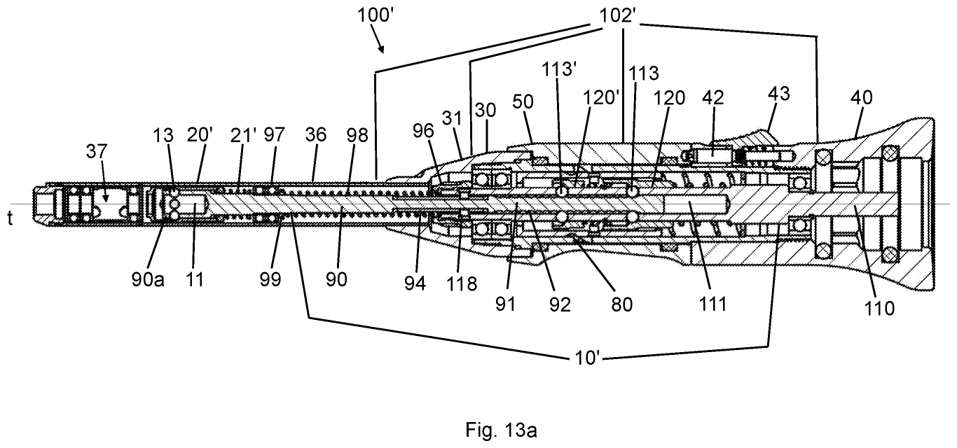

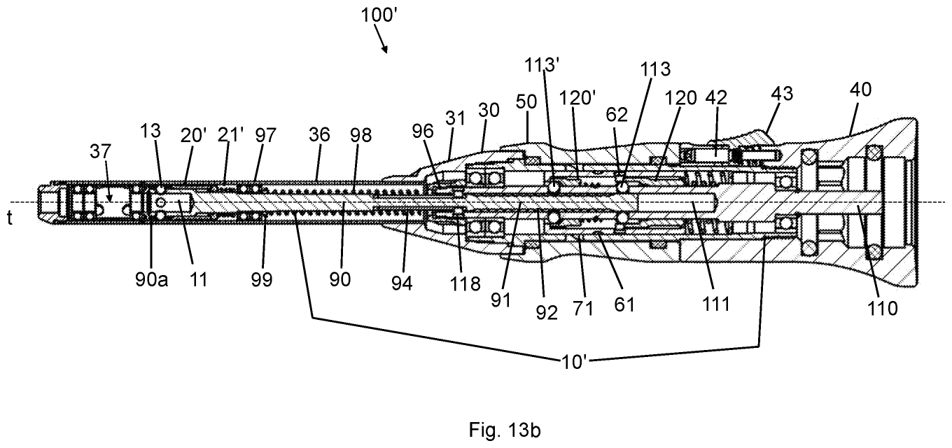

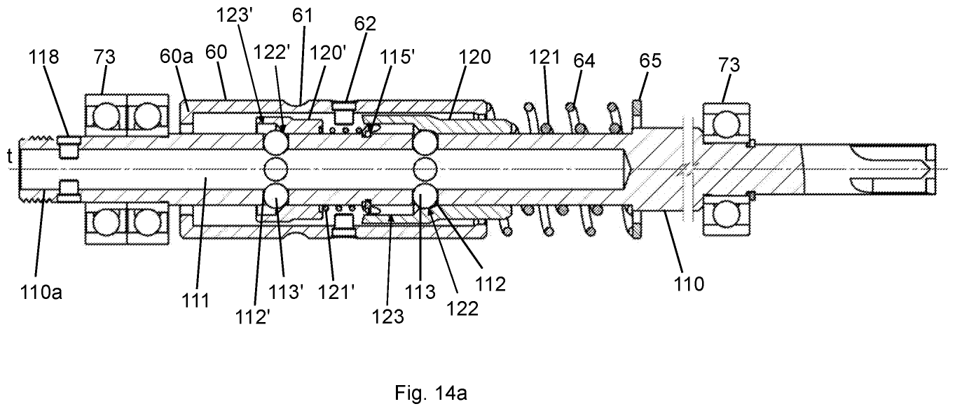

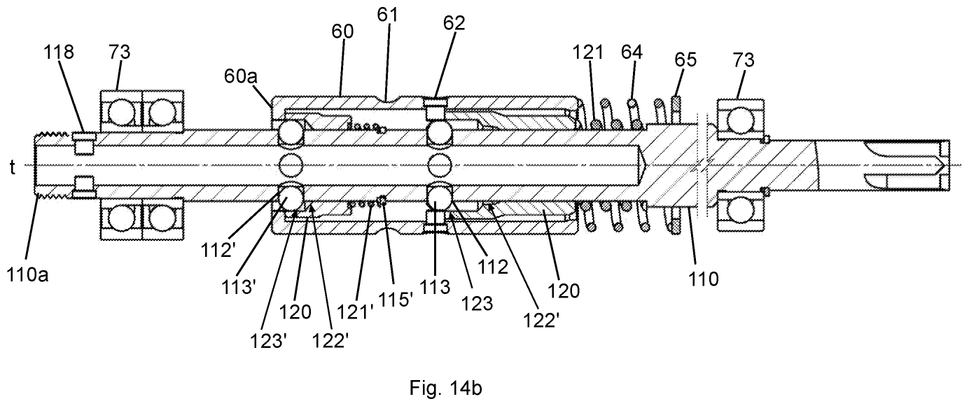

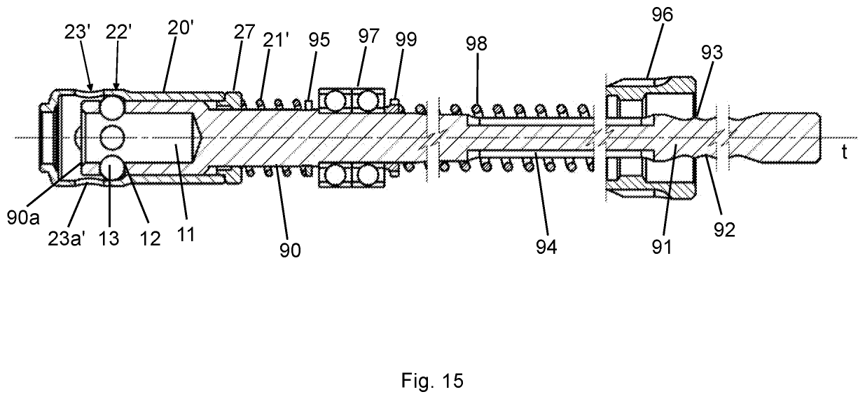

Further details of the invention will be described by way of exemplary embodiments with reference to the drawings, wherein:
FIG. 1 is a cross-sectional view of a first embodiment of a handpiece according to the invention;
FIG. 1a is an enlarged image of the detail A of FIG. 1 in the closed position of the handpiece;
FIG. 1b is an enlarged image of the detail A of FIG. 1 in the open position of the handpiece;
FIGS. 2a-2b are each a perspective view of the ball retainer of the handpiece according to FIG. 1;
FIG. 3 is an exploded perspective view of the main components of the handpiece according to FIG. 1;
FIG. 4 is an exploded perspective view of the drive shaft and opening bushing of the handpiece according to FIG. 1;
FIG. 5 is an exploded perspective view of the rotary ring and bearing housing of the handpiece according to FIG. 1;
FIG. 6 is an exploded perspective view of the handle of the handpiece according to FIG. 1;
FIG. 7 is a perspective view of the rotary ring;
FIG. 8 is a cross-sectional view of the handle;
FIG. 9 is a sectional view of the nose portion of the handpiece according to FIG. 1;
FIG. 10a is a cross-sectional view illustrating the relative position of the drive shaft and the opening bushing when the handpiece is in closed position;
FIG. 10b is a cross-sectional view illustrating the relative position of the drive shaft and the opening bushing when the handpiece is in open position;
FIG. 11 is a side view of an exemplary embodiment of a rotary tool according to the invention;
FIG. 11a is a side view of a part of the stem of the rotary tool according to FIG. 11;
FIG. 12 is a side view of a part of the stem of another exemplary rotary tool;
FIG. 13a is a cross-sectional view of a second embodiment of the handpiece according to the invention in the closed position;
FIG. 13b is a cross-sectional view of the second embodiment of the handpiece according to the invention in the open position;
FIG. 14a is a cross-sectional view illustrating the relative position of the shaft body and the opening bushing in the closed position of the second embodiment;
FIG. 14b is a cross-sectional view illustrating the relative position of a shaft body and the opening bushing in the open position of the second embodiment;
FIG. 15 is a cross-sectional view of the shaft extension of the second embodiment.

### FIRST EMBODIMENT

FIGS. 1-10 illustrate a preferred embodiment of a surgical handpiece 100 according to the invention and its components. The surgical handpiece 100 comprises a housing 102 and a drive shaft 10 arranged therein, driven by a motor not shown in the figures. In the drive shaft 10 a stem-engaging bore 11 is provided having a longitudinal axis t in common with the drive shaft 10, and which is open at a distal end 10a of the drive shaft 10. In the wall of the drive shaft 10, along its circumference perpendicular to the longitudinal axis t, flanged bores 12 are formed, whose flanges 12a are on the inner side of the wall of the drive shaft 10 facing the stem-engaging bore 11, as can be seen in the enlarged view of the part marked A in FIG. 1 a. A fixing ball 13 is fitted in each of the flanged bores 12. At the outer side of the wall of the drive shaft 10 a ball retainer 20 is provided which is movable between closed and open positions. Said ball retainer 20 is arranged in the closed position to wedge the fixing balls 13 into the flanged bore 12, as shown in FIGS. 1 and 1a, and in the open position to allow the partial exit of the fixing balls 13 from the flanged bore 12 as shown in FIG. 1b.

In the present embodiment, there are four flanged bores 12 and four balls 13. In other embodiments, there may be more or fewer flanged bores 12 and balls, but at least three flanged bores 12 and balls 13. The appropriate number of these is determined by the diameter of the stem-engaging bores 11 and the diameter of the balls 13. Preferably, the dimensions and numbers are selected such that in the closed position of the ball retainer 20 the distance between adjacent fixing balls 13 is less than 0.5 mm, preferably less than 0.2 mm, more preferably less than 0.1 mm.

In the present embodiment, the ball retainer 20 is configured as a sleeve that surrounds the drive shaft 10 and is slidable along the drive shaft against a spring 21. The end of the spring 21 opposite the ball retainer 20 rests on a shoulder 15 formed on the drive shaft 10, which is shown in FIGS. 2a and 2b. A first inner diameter section of the ball retainer 20 forms a closing section 22, and a second larger inner diameter section functions as an opening section 23. In the closed position of the ball retainer 20, the closing section 22 of the ball retainer 20 is adjacent to the flanged bores 12 (see FIG. 1a), and in the open position of the ball retainer 20 the opening section 23 is adjacent to the flanged bores 12 (see FIG. 1b). The inner diameters of the closing section 22 and the opening section 23 may vary, in which case the first and second diameters are defined as the smallest inner diameters.

The inner diameter of the closing section 22 is chosen so that the closing section 22 is at a distance from the outside of the drive shaft 10 wall that will allow the balls 13 to be forced into the flanged bore 12 (see FIG. 1a). The inner diameter of the opening section 23 is chosen so that the opening section 23 is at a distance from the outside of the drive shaft 10 wall that will allow the balls 13 to partially exit the flanged bore 12 (see FIG. 1b).

Not only the depicted sleeve-shaped ball retainer 20 can be conceived. It is also possible to provide an embodiment in which the ball retainer 20 is a plurality of elongated profiles which are displaceable along the drive shaft 10, the closing section 22 of which is less far from the wall of the drive shaft 10 than the opening section 23. A ball retainer 20 operating on a completely different principle may also be used, for example, the ball retainer 20 may comprise radially movable jaws each of which may be clamped onto a single ball 13.

The ball retainer 20 is preferably connected to the drive shaft 10 in a rotation-free manner. In the present embodiment, this is achieved by providing bores 24 in the wall of the ball retainer 20 in a circle (see FIGS. 2a-2b) , and providing apertures 14 parallel with the direction of the longitudinal axis t in the wall of the drive shaft 10 (see FIG. 4), and the bores 24 of the ball retainer 20 and the longitudinal apertures 14 in the drive shaft 10 are connected by pins 26 (see FIG. 4), so that the ball retainer 20 cannot rotate relative to the drive shaft 10 along the longitudinal axis t, but the pins 26 can move along the longitudinal aperture 14, thus allowing the ball retainer 20 to slide along the drive shaft 10 between the closed and open position.

Preferably, the ball retainer 20 has a profile containing grooves 25. These can be used to lock the ball retainer 20 against rotation, and also the drive shaft 10, as will be discussed later.

The main elements of the exemplary handpiece shown in FIG. 1 are illustrated in the exploded view in FIG. 3.

In the present embodiment, the housing 102 includes a nose portion 30, a handle 40 and a rotary ring 50 rotatable relative thereto about the longitudinal axis t between first and second positions. An opening bushing 60 and a bearing housing 70 are arranged concentrically around the drive shaft 10 and fixed against rotation within the housing 102. The individual components shown in exploded view in FIG. 3 are further detailed in further exploded views: the opening bushing 60 and the components located around the shaft 10 in FIG. 3 are shown in exploded view in FIG. 4, the rotary ring 50 and bearing housing 70 are shown in exploded view in FIG. 5, and the handle is shown in exploded view in FIG. 6.

FIG. 7 shows the rotary ring 50 separately. On the inner side of the wall of the rotary ring 50, there is at least one groove 51 (in this embodiment, two opposite grooves 51) running parallel to the longitudinal axis t. Preferably, the outer side of the rotary ring 50 has an ergonomically shaped gripping surface 52. In the present embodiment, there is a first bore 53 and a second bore 54 at the proximal end 50b towards the grip 40 of the rotary ring 50, which preferably have different depths. Rotation of the rotary ring 50 about the bearing housing 70 is facilitated by Teflon bushings 56 provided in grooves 55 in the interior of the rotary ring 50 along a circumference perpendicular to the longitudinal axis t, which can be observed in FIGS. 1 and 5.

In the wall of the bearing housing 70, at least one (in the present embodiment, two opposing) helical bore 71 is provided, and on the outer side of the wall of the opening bushing 60, a circumferential groove 61 is provided perpendicular to the longitudinal axis t. The rotating ring 50, the bearing housing 70 and the opening bushing 60 are connected by at least one positioning ball 80, which extends through the spiral bore 71 of the bearing housing 70 such that one side of the positioning ball 80 extends into the longitudinal groove 51 of the rotating ring 50 which groove 51 is parallel with the longitudinal axis t, and the other side of the positioning ball 80 extends into the circumferential groove 61 of the opening bushing 60. In this way, in the first position of the rotating ring 50, the opening bushing 60 is in a first position along the longitudinal axis t, as shown in FIG. 1a, and in the second position of the rotating ring 50, the opening bushing 60 is in a second position along the longitudinal axis t, offset from the first position, as shown in FIG. 1b.

The opening bushing 60 has a stop 62 cooperating with the ball retainer 20, which is moved away from the ball retainer 20 in the first position of the opening bushing 60, thereby allowing the spring 21 to force the ball retainer 20 into the closed position (see FIG. 1a). In the second position of the opening bushing 60, the stop 62 abuts the ball retainer 20 and, due to the offset position of the opening bushing 60, forces the ball retainer 20 into the open position against the spring 21.

The stop 62 is preferably configured to engage with the groove 25 of the ball retainer 20 in the second position of the opening bushing 60, thereby preventing rotation of the ball retainer 20 and, thus, rotation of the drive shaft 10. This has a safety function, as will be discussed later. In the present embodiment, the stop 62 comprises a plurality of pins arranged along a circumference perpendicular to the longitudinal axis t, which are guided through bores 63 of the opening bushing 60 (see FIG. 4).

Preferably, a second spring 64 is arranged around the drive shaft 10 to force the opening bushing 60 into the first position, which in this embodiment is supported on a spring plate 65 arranged around the shaft 10. The spring plate 65 is supported by a circumferential shoulder 72 located between the sections of the bearing housing 70 of different inner diameters (see FIG. 1).

Within the bearing housing 70, a ball bearing system 73 is preferably arranged at a plurality of locations to allow rotation of the drive shaft 10 within the bearing housing 70. The distal ball bearing system 73 is held in place by a nut 74 screwed onto the threaded distal end 10a of the drive shaft 10. The proximal bearing system 73 is retained by a retaining ring 17 fitted in a circumferential groove 16 of the drive shaft 10.

In the present embodiment, bearing housing 70 has two threaded ends 75a, 75b. The distal threaded end 75a is screwed into the nose portion 30, and the proximal threaded end 75b is screwed into the handle 40.

The opening bushing 60 is fixed in the bearing housing 70 in such a way that it cannot rotate while being displaced along the longitudinal axis t. In the present embodiment, this is ensured by grooves 66 in the wall of the opening bushing 60 along the longitudinal axis t (see FIG. 4) and by pins 78 passing through bores 77 in the bearing housing 70, which extend into the grooves 66 and prevent rotation of the opening bushing 60 and the bearing housing 70 relative to each other.

The structure of the handle 40 is best observed in the exploded view of FIG. 6 and in the sectional view of FIG. 8.

In the vicinity of the distal end 40a of the handle 40 adjacent the rotary ring 50, there is an opening coupling shaft 42 movable against a third spring 41, a distal end 42a of which is forced into the first bore 53 in the first position of the rotary ring 50 and into the second bore 54 in the second position of the rotary ring 50 by the third spring 41.

The distal end 42a of the opening coupling shaft 42 is preferably rounded, and the depth of the first bore 53 is dimensioned to lock the rotary ring 50 against rotation when the spring 41 is forced into the distal end 42a of the opening coupling shaft 42. The depth of the second bore 54 is dimensioned such that the rounded distal end 42a exits the second bore 54 under the action of a torque applied to the rotary ring 50 in excess of a given threshold. Preferably, the depth of the first bore 53 is at least 1 mm and the depth of the second bore 54 is at most 0.2 mm.

The opening coupling shaft 42 can be moved in the proximal direction against the spring 41 by means of an opening switch 43. For example, a threaded portion 42b of the opening coupling shaft 42 is threaded into a threaded bore 45 formed on a perpendicular face 44 of the opening switch 43.

In the handle 40, as is known per se, flexible O-rings 46 are provided to absorb vibrations of the motor arranged herein, which is not shown.

In the present embodiment, the internal structure of the nose portion 30 is shown in FIG. 9. The nose portion 30 has an outer shell 31. Within this, a ball bearing system 32 is provided to allow support of a stem fixed in the stem-engaging bore 11 while providing easy rotation in a known manner. The ball bearing system 32 is held at the desired distance from the distal ball bearing system 73 of the bearing housing 70 by a bearing spacer 33. In the present embodiment, a straight spacer 34 and a corrugated spacer 35 are provided between the ball bearing system 32 and the bearing spacer 33.

In some cases, it may be necessary to insert an additional, not shown, cover element in a slot 81 between the nose portion 30 and the rotary ring 50. In the present embodiment, this can be achieved by a Duraguard fixing slotted leaf spring 82 and balls 83, which are inserted into bores 76 of the bearing housing 70, where they are engaged by the shell 31 of the nose portion 30 on one side and the slotted spring 82 on the other side. The distal end 60a of the opening bushing 60 is configured so that in the first (closing) position of the rotary ring 50, it slides under the slotted spring 82 as shown in FIG. 1a and is radially biased outward, thereby also displacing the balls 83 outward and securing an additional covering element inserted in an opening 81 (FIG. 1a and FIG. 10a). In the second (opening) position of the rotary ring 50, an end 60a of the opening bushing 60 comes out from under the slotted spring, allowing the balls 83 to be pushed radially in and the additional covering element to be freely inserted and removed (FIG. 1b and FIG. 10b).

### Rotary Tool

A stem 202 of a rotary tool 200 according to the invention may be inserted into the stem-engaging bore 11 of the drive shaft 10. FIG. 11 illustrates an exemplary rotary tool 200 having a longitudinal axis t′ and having a stem 202 and a surgical head 204. The surgical head 204 may be of any configuration, such as a drill head, milling head, ball milling head, diamond milling head, twist drill head, etc. In the embodiment shown in FIG. 11, the surgical head 204 is a craniotome blade.

The stem 202 is formed with at least three ball receiving recesses 205 along a circumference perpendicular to its longitudinal axis t. The number of recesses 205 corresponds to the number of flanged bores 12 formed in the drive shaft 10 of the given handpiece 100 and the number of fixing 13 balls inserted therein. The rotary tool 200 shown in FIG. 11 has four recesses, which are also shown enlarged in FIG. 11a. This rotary tool 200 can be fitted into the handpiece 100 shown in FIG. 1. The recesses 205 are preferably formed as notches. The smallest width of an edge 206 parallel to the longitudinal axis t′ between the adjacent ball receiving recesses 205 is less than 0.5 mm, preferably less than 0.2 mm, more preferably less than 0.1 mm, i.e. the notches created on the stem 202 are preferably practically touching each other. The portion of the stem 202 of the tool 200 of FIG. 11 including four recesses 205 is shown enlarged in FIG. 11a.

FIG. 12 shows a detail of a rotary tool 200 according to the invention, having a stem 202 with three recesses 205 formed along a circumference perpendicular to the longitudinal axis t′. Such a rotary tool 200 may be inserted into a handpiece 100, which also comprises three flanged bores 12 and a total of three fixing balls 13 are inserted therein.

The radius of curvature of the recesses 205 is preferably the same as the radius of curvature of the fixing balls 13, and the recesses 205 are dimensioned so that, when the stem 202 of the rotary tool 200 is inserted into the stem-engaging bore 11 and the ball retainer 20 is brought to the closed position, the fixing balls 13, which are inserted into the flanged bores 12 and which extend through the flanged bores 12 into the stem-engaging bore 11, completely fill the recesses 205. This means that the depth of the recesses 205 is the same as the thickness of the piece of each fixing ball 13 that extends beyond the flanged bore 12 when the fixing ball 13 is forced by the ball retainer 20 into each flanged bore 12 in the closed position of the ball retainer 20. Thickness here means the height of the piece (i.e., the spherical slice) extending beyond the flanged bore 12.

Preferably, the stem-engaging bore 11 of the drive shaft 10 and the stem 202 are configured such that the stem 202 can be inserted into the bore 11 at a depth such that the recesses 205 are aligned with the flanged bores 12 perpendicular to the longitudinal axis t′.

### Operation of the First Embodiment

In the following, the use of the handpiece 100 (shown in FIGS. 1 to 10) and the rotary tool 200 that can be fitted into it is discussed.

At the start of use, the rotary tool 200 is not yet inserted into the handpiece 100, which is in a closed position, as illustrated in FIG. 1, FIG. 1a and FIG. 10a. At this time, the rotary ring 50 is in its first position, that is, the opening coupling shaft 42 of the handle 40 is in the first bore 53 formed in the proximal end 50b of the rotary ring 50 by the third spring 41. The balls 80 connecting the rotary ring 50, the opening bushing 60 and the bearing housing 70 are in a distal position within the longitudinal grooves 51 of the rotary ring 50 and within the helical bores 71 of the bearing housing 70, and therefore the opening bushing 60 is also in its distally offset first position. The ball retainer 20 is held in the closed position by the first spring 21, i.e. the closing section 22 of the ball retainer 20 is positioned above the flanged bores 12 of the drive shaft 10, and the closing section 22 forces the balls 13 into the flanged bore 12, as can be seen in FIG. 10a showing the elements adjacent to the drive shaft 10. In such a case, the opening bushing 60 is held in the open position by the spring 64, so that the stop 62 is moved away from the ball retainer 20.

To insert the rotary tool 200, the user puts the handpiece 100 in the open position. To do so, the opening switch 43 on the handle 40 is pulled back in a proximal direction, causing the distal end 42a of the opening coupling shaft 42 to exit the first bore 53 of the rotary ring 50. The user then rotates the rotary ring 50 to the second position. The gripping surface 52 assists in gripping and rotating the rotary ring 50. In the second position, the second bore 54 at the proximal end 50b of the rotary ring 50 is aligned with the opening coupling shaft 42. In this position, the third spring 41 automatically pushes the opening coupling shaft 42 back in the distal direction, causing the distal end 42a of the opening coupling shaft 42 to enter the second bore 54, whereby the rotary ring 50 is locked in the second position. In the absence of the third spring 41, the user could manually perform the locking by pushing back the opening switch 43, but it is preferable from an accident prevention point of view that the locking of the open position is performed automatically.

As the rotary ring 50 rotates from the first position to the second position, each ball 80 tracks the angular rotation of the associated longitudinal groove 55 parallel with the longitudinal axis t, by moving in the corresponding angular displacement in the helical bore 71 in the bearing housing 70 and in the circumferential groove 61 in the opening bushing 60. Meanwhile, the ball 80 is also displaced along the longitudinal axis t due to the helical bore 71. As a result, the ball 80 pushes the opening bushing 60 in a proximal direction against the second spring 64 by engaging the circumferential groove 61, and causes the opening bushing 60 to move to the second position illustrated in FIGS. 1b and 10b. During the displacement of the opening bushing 60, the stop 62 enters the groove 25 of the ball retainer 20 and pushes the ball retainer 20 into the open position against the first spring 21. The stop 62 plays a role not only in displacing the ball retainer 20, but also in locking it against rotation. The opening bushing 60 is rotationally locked in the bearing housing 70. By coupling the stop 62 of the opening bushing 60 to the groove 25 of the ball retainer 20, the ball retainer 20 can no longer rotate relative to the opening bushing 60. The ball retainer 20 is also non-rotatably connected to the drive shaft 10 by the pins 26, so that the stop 62, together with the ball retainer 20, also blocks rotation of the drive shaft 10 relative to the bearing housing 70. This has a safety function; the user cannot inadvertently activate the rotation of the drive shaft 10 when inserting the rotary tool 200.

When the ball retainer 20 is in the open position, the opening section 23 is positioned over the flanged bores 12 of the drive shaft 10, allowing the balls 13 to exit the flanged bores 12, freeing the stem-engaging bore 11.

The user then inserts the stem 202 of the rotary tool 200 into the stem-engaging bore 11(not shown). Finally, the user returns the handpiece 100 to the closed position to lock the rotary tool 200. This can be done, on the one hand, by pulling back the opening switch 43 on the handle 40 in the proximal direction and rotating the rotary ring 50 to the first position. On the other hand, in this preferred embodiment, it is also possible to perform the closing of the handpiece 100 by the user by rotating the rotary ring 50 back to the first position without retracting the opening coupling shaft 42. This is made possible by the shallow depth of the second bore 54 and the rounded end 42a of the opening coupling shaft 42. The rounded end 42a is pushed out of the bore 54 by the edge of the shallow bore 54 under the torque applied to the rotary ring 50. Upon reaching the first position of the rotary ring 50, the third spring 41 automatically pushes the opening coupling shaft 42 back into the first bore 53, which locks the rotary ring 50 in place.

When the rotary ring 50 is rotated to the first position, the opposite process to the one described above takes place. During rotation of the rotary ring 50, the helical bore 71 of the bearing housing 70 guides the ball 80 back to the distal position in the longitudinal groove 51 parallel with the longitudinal axis t, while the ball 80 pushes the opening bushing 60 back in the distal direction, by means of the circumferential groove 61, to the first position shown in FIGS. 1a and 10a. By removing the stop 62, the spring 21 also pushes the ball retainer 20 back into the closed position. In this manner, the closing section 22 is positioned over the flanged bores 12 and forces the balls 13 into the flanged bores 12 and simultaneously into the circumferential recesses 205 formed in the stem 202 of the rotary tool 200. Due to the fact that in the closed position of the ball retainer 20 the distance between the adjacent fixing 13 balls is less than 0.5 mm, preferably less than 0.2 mm, more preferably less than 0.1 mm, and accordingly the smallest width of the edges 206 between the recesses 205 on the stem 202 is less than 0.5 mm, preferably less than 0.2 mm, more preferably less than 0.1 mm, the balls 13 run off the narrow edges 206 and automatically rotate the rotary tool 200 into the position at which each recess 205 is located on the inner side of each flanged bore 12. When the ball retainer 20 is in the closed position, the fixing balls 13, which are forced into the flanged bore 12 by the closing section 22 and extend through the flanged bore 12 into the stem-engaging bore 11, completely fill the recesses 205 and secure the rotary tool 200 firmly.

The connection with the balls 13 is not only used to hold the stem 202, but also to transmit the torque of the drive shaft 10 during operation. This results in a simpler and easier to use structure compared to the state of the art.

### SECOND EMBODIMENT

FIGS. 13-15 show another preferred embodiment. The same elements of the handpiece 100′ according to the second embodiment compared to the first embodiment have been provided with the same reference numerals. The structure and operation of the handle 40, the rotary ring 50, the opening bushing 60 and the bearing housing 70 are essentially the same as those shown in the first embodiment and will therefore not be described in detail. In the following, the differences with respect to the first embodiment will be detailed.

A handpiece 100′ according to the second embodiment, comprising a housing 102′ and a drive shaft 10′ arranged therein, is shown in the closed position in FIG. 13a and in the open position in FIG. 13b. In this embodiment, the length of the drive shaft 10′ is variable owing to the fact that the drive shaft 10′ comprises a proximal shaft body 110 and a distal shaft extension 90, which are adapted to be fixed to each other at different distances along the longitudinal axis t.

The proximal stem 91 of the shaft extension 90 can be engaged at different depths in a second stem-engaging bore 111 opening from a distal end 110a of the shaft body 110. For the engagement, the shaft body 110 is provided with a ball retaining mechanism similar to that of the first embodiment. At least three (in this case four) second flanged bores 112 are formed in the wall of the shaft body 110 along a circumference perpendicular to the longitudinal axis t, opening into the second stem-engaging bore 111, the flanges 112a of the second flanged bores 112 being located on the inner side of the wall of the shaft body 110 adjacent the second stem-engaging bore 111. A second fixing ball 113 is inserted into each of the second flanged bores 112. There is a second ball retainer 120 movable between closed and open positions on the outer side of the wall of the shaft body 110. The second ball retainer 120, in the closed position, forces the second balls 113 into the second flanged bore 112 and, in the open position, allows the second balls 113 to partially exit the second flanged bore 112. The ball retainer 120 has a closing section 122 and an opening section 123 which can be displaced against a spring 121. In the closed position of the ball retainer 120 the closing section 122 is adjacent to the flanged holes 112 (see FIG. 1.3.1). In its open position, the opening section 123 is adjacent to the flanged bores 112 (see FIG. 14b). The arrangement, mounting and operating mechanism of the ball retainer 120 is identical to that of the ball retainer 20 shown in the first embodiment. Accordingly, the ball retainer 120 is also non-rotatably coupled to the shaft body 110 and has a profile cooperating with the stop 62 of the opening bushing 60, which is coupled to the stop 62 in the second position of the opening bushing 60 (see FIG. 14b), and prevents rotation of the ball retainer 120 and also of the shaft body 110 relative to the opening bushing 60, which is itself rotationally fixed within the bearing housing 70, as shown in the first embodiment.

In FIG. 14a corresponding to the open position of the handpiece 100′ and in FIG. 14b corresponding to the closed position, it can be seen that an additional ball retainer 120′ is arranged along the shaft body 110. At least three (in this case four) additional flanged bores 112′ are formed in the wall of the shaft body 110 along an other circumference perpendicular to the longitudinal axis t, opening into the second stem-engaging bore 111, into each of which a further fixing ball 113′ is also inserted. The ball retainer 120′ is also configured as a sleeve that encircles the drive shaft 110 and is slidable along its length against a spring 121′. An end of the spring 121′ opposite to the ball retainer 120′ rests on a retaining ring 115′ arranged on the drive shaft 110. A first inner diameter section of the ball retainer 120′ forms a closing section 122′ and a second larger inner diameter section functions as an opening section 123′. In the closed position of the ball retainer 120′, the closing section 122′ of the ball retainer 120′ is adjacent to the flanged bores 112′ (see FIG. 14a), and in the open position, the opening section 123′ of the ball retainer 120′ is adjacent to the flanged bores 112′ (see FIG. 14b). To create the open position of the ball retainer 120′, a distal edge 60a of the opening bushing 60 is used, which abuts the ball retainer 120′ during the proximal displacement of the opening bushing 60 and pushes the opening section 123′ over the flanged bore 112′.

The stem 91 of the shaft extension 90 comprises a plurality of parallel grooves 92 formed along a circumference perpendicular to the longitudinal axis t for partially receiving the fixing balls 113, 113′, as better observed in FIG. 15. The radius of curvature of the circumferential grooves 92 is the same as the radius of curvature of the balls 113, 113′. Preferably, the edges 93 separating the circumferential grooves 92 are extremely narrow, with a width along the longitudinal axis t preferably less than 0.5 mm, preferably less than 0.2 mm, more preferably less than 0.1 mm, whereby the balls 113, 113′ run off the edges and into the nearest groove 92 when clamped. The spacing of the flanged bores 112, 112′ is dimensioned to be an integer multiple of the width of the circumferential grooves 92, so that each ball 113, 113′ is seated simultaneously in a single circumferential groove 92, as can be seen in FIG. 13a. The double circumferential anchorage formed by the balls 113 and 113′ results in a more stable clamping of the stem 91. Of course, more or fewer ball retainers 120, 120′ and associated balls 113, 113′ can also be used to clamp the stem 91.

The shaft body 110 and the shaft extension 90 comprise interconnected elements preventing their rotation relative to each other, which in the present embodiment are longitudinal grooves 94 formed on the shaft extension 90 parallel with the longitudinal axis t, and pins 118 extending through the wall of the shaft body 110 and adapted to fit into the grooves 94. Thus, in this embodiment, the rotational movement of the shaft body 110 is transmitted by the pins 118 to the shaft extension 90 through the longitudinal grooves 94.

In the present embodiment, the stem-engaging bore 11 opens from the distal end 10a of the drive shaft 10, which in this embodiment is also the distal end 90a of the shaft extension 90. The stem-engaging bore 11 is provided with a ball-type retaining and torque-transmitting mechanism as well wherein at least three (in the present case four) flanged bores 12 opening into the stem-engaging bore 11 are formed in the wall of the shaft extension 90 along a circumference perpendicular to the longitudinal axis t, and a fixing ball 13 is inserted in each of the flanged bores 12. On the outer side of the wall of the shaft extension 90 there is a ball retainer 20′ movable between a closed and an open position, which ball retainer 20′, in the closed position, forces the balls 13 into the flanged bores 12 and, in the open position, allows the partial exit of the fixing balls 13 from the flanged bores 12. The ball retainer 20′ has a closing section 22′ and an opening section 23′ and can be displaced from the closed position to the open position against a spring 21′ along the shaft extension 90. In the closed position the closing section 22′ of the ball retainer 20′ is adjacent to the flanged bore 12 and in the open position the opening section 23′ of the ball retainer 20′ is adjacent to the flanged bore 12. When the ball retainer 20′ is in the closed position, the distance between the adjacent fixing balls 13 is less than 0.5 mm, preferably less than 0.2 mm, more preferably less than 0.1 mm. The ball retainer 20′ is also sleeve-shaped in the present embodiment, the opening section 23′ of which is provided with bores 23a allowing partial exit of the balls 13. The shaft extension 90 and the ball retainer 20′ are surrounded by a tube 36 inserted into the shell 31 of the nose portion 30, which prevents the balls 13 from completely exiting from the flanged bores 12 through the bores 23a. The ball retainer 20′ is covered by a cover 27 in the proximal direction, which is open in the middle and through which the shaft extension 90 is passed. The spring 21′ is supported on the distal side by the cover 27 and on the proximal side by a retaining ring 95, which is fixed in a non-displaceable manner on the shaft extension 90 (see FIG. 15).

In this embodiment, a bearing nut 96 shown in FIG. 15 secures the shaft body 110 to the shaft extension 90, such that the bearing nut 96 is driven onto the threaded distal end 110a of the shaft body 110 and extends over the pins 118, retaining them in the grooves 94 (see FIGS. 13a and 13b).

In this embodiment, two ball bearing systems 97 are arranged within the tube 36 to provide unobstructed rotation of the shaft extension 90 within the tube 36. The number of ball bearing systems 97 used is preferably adapted to the length of the shaft extension 90 and the length of the tube 36.

A spring 98 is arranged between the bearing nut 96 and the ball bearing system 97 for pushing the shaft extension 90 in the distal direction, which spring 98 rests on a spring plate 99 arranged next to the ball bearing system 97. The spring 98 has a preferably higher spring constant than the spring 21′, i.e., a higher force is required to compress the spring 98, so that the spring 21′ compresses sooner, whereby the ball retainer 20′ is always brought to the closed position when the handpiece 100′ is moved to the closed position.

From the distal direction, the shaft extension 90 is bounded by a nose bearing unit 37 fixed in the tube 36, in which preferably further ball bearing systems and one or more spacers therebetween can be arranged to ensure unobstructed rotation of the rotary tool 200. The shaft extension 90 is dimensioned such that, in the closed position, the distal end 90a is spaced from the nose bearing unit 37 to such an extent that even the ball retainer 20′, in the closed position, which is displaced in the distal direction by the spring 21′, is spaced from the nose bearing unit 37 even when the balls 113 extend into the proximally most distal groove 92 of the shaft extension 91 of the shaft 90, as shown in FIG. 13a. However, when the handpiece 100′ is in the open position, the spring 98 moves the shaft extension 90 distally the spring plate 99 and the ball bearing system 97 so far, that its distal end 90a abuts the nose bearing unit 37 or the portion of the ball retainer 20′ between the nose bearing unit 37 and the distal end 90a, as can be seen in FIG. 13b. If no nose bearing unit 37 is used, the distal end of the tube 36 may also be provided with an abutting flange. In the distal forward position of the shaft extension 90, the flanged bores 12 are also distally offset from the ball retainer 20′, as it also abuts the nose bearing unit 37, and thus cannot move distally by the same extent as the shaft extension 90. For this reason, the flanged bores 12 are positioned adjacent to the openings 23′ of the ball retainer 20′, i.e. this will be the open position of the ball retainer 20′.

### Operation of the Second Embodiment

The use of the handpiece 100′ of the second embodiment with the rotary tool 200 of the invention is as follows. The differences with respect to the first exemplary embodiment will be discussed in detail when describing the use.

When not in use, the handpiece 100′ is in the closed position shown in FIG. 13a, and the rotary tool 200 is not yet inserted in the handpiece 100′. As described for the first embodiment, in this case, the rotary ring 50 is in its first position, the balls 80 coupling the rotary ring 50, the opening bushing 60 and the bearing housing 70 are in a distal position within the longitudinal grooves 51 of the rotary ring 50 and within the helical bores 71 of the bearing housing 70, and therefore the opening bushing 60 is also in its distally offset first position. In the first position of the opening bushing 60, the stop 62 and the distal edge 60a′ of the opening bushing 60 are spaced from the ball retainer 120 and the ball retainer 120′, respectively. In such a case, the ball retainers 120 and 120′ are held in the closed position by the springs 121 and 121′, as can be observed in FIG. 14a. The balls 113 and 113′ extend through the flanged bores 112 and 112′ into each of the circumferential grooves 92 in the stem 91 of the shaft extension 90, and secure the shaft extension 90 against displacement along the longitudinal axis t. In this way, the distal end 90a of the shaft extension 90 is spaced from the nose bearing unit 37, so that the spring 21′ can hold the ball retainer 20′ in the closed position, i.e. the balls 13 are forced into the flanged bores 12 opening into the stem-engaging bore 11.

To insert the rotary tool 200, the user puts the handpiece 100′ in the open position. To do this, the opening switch 43 on the handle 40 is pulled back in a proximal direction, causing the distal end 42a of the opening coupling shaft 42 to exit the first bore 53 of the rotary ring 50. Subsequently, the user rotates the rotary ring 50 to the second position, where the distal end 42a of the opening coupling shaft 42 enters the second bore 54, whereby the rotary ring 50 is locked in the second position.

As the rotary ring 50 rotates from the first position to the second position, each ball 80 tracks the angular rotation of the associated groove 55 in the longitudinal direction t. By virtue of the helical bore 71, the ball 80 also moves along the longitudinal axis t. As a result, the ball 80 also moves the opening bushing 60 in the proximal direction via the circumferential groove 61 against the second spring 64, and causes the opening bushing 60 to move to the second position illustrated in FIGS. 13b and 14b. During the movement of the opening bushing 60, the stop 62 and the edge 60a′ push the ball retainers 120 and 120′ into the open position against the springs 121 and 121′. The stop 62 rotationally locks the ball retainer 120 against rotation, and also the shaft body 110, as detailed in relation to the first embodiment.

When the ball retainers 120 and 120′ are in the open position, the opening sections 123 and 123′ are positioned over the flanged bores 112 and 112′ of the shaft body 110, so that the balls 113 can exit radially from the flanged bores 112 and 112′. The spring 98 pushes the shaft extension 90 in a distal direction by the spring plate 99. As a result, the circumferential grooves 92 guide the balls 113 and 113′, and the balls 113 and 113′ are ejected from the grooves 92, which is enabled by the fact that the balls 113 and 113′ can partially exit the flanged bores 112 and 112′ in the open position of the ball retainers 120 and 120′. The spring 98 pushes the shaft extension 90 in the distal direction until the distal end 90a abuts the nose bearing unit 37, which in this case is via the distal end of the ball retainer 20′. In this way, the ball retainer 20′ is moved to an open position, i.e. the distal movement of the shaft extension 90 causes the opening section 23′ of the ball retainer 20′ to be positioned adjacent to the flanged bores 12, thus allowing the balls 13 to exit the flanged bores 12.

Then, with the handpiece 100′ in the open position, the user pushes the stem 202 of the rotary tool 200 into the stem-engaging bore 11 of the shaft extension 90 through the tube 36 and its nose bearing unit 37. The stem 202 forces the balls 13 to exit the stem-engaging bore 11 through the flanged bores 12. In this embodiment, the depth of the stem-engaging bore 11 is selected to correspond to the distance of the recess 205 formed on the stem 202 of the rotary tool 200 from the proximal end of the stem 202. In this way, when the proximal end of the stem 202 reaches the bottom of the bore 11, the recesses 205 are aligned with the balls 13. By pushing the rotary tool 200 further inward, the stem 202 displaces the shaft extension 90 in the proximal direction. Meanwhile, the spring 21 continues to push the ball retainer 20′ in the distal direction, causing it to move in the distal direction relative to the flanged bores 12 of the shaft extension 90 (in fact, the shaft extension 90 moves relative to the ball retainer 20′). This causes the closing section 22′ of the ball retainer 20′ to be positioned adjacent to the flanged bores 12, forcing the balls 13 into the flanged bores 12 and into the aligned recesses 205 in the stem 202. Due to the narrow edges 206, the balls 13 automatically rotate the stem 202 into a position in which the recesses 205 are also radially aligned with the flanged bores 12. Then, the balls 13 lock the stem 202 of the rotary tool 200 in the stem-engaging bore 11. The user can continue to offset the shaft extension 90 in the proximal direction to the desired depth by further inserting the rotary tool 200 in order to control the extent to which the rotary tool 200 protrudes distally from the tube 36 of the handpiece 100′. When the desired extent of protrusion is achieved, the user closes the 100′ handpiece again to lock the shaft extension 90 along the longitudinal axis t. This can be done, on the one hand, by pulling back the opening switch 43 on the handle 40 in the proximal direction and rotating the rotary ring 50 to the first position. On the other hand, as described in the first embodiment, it may be performed by bringing the handpiece 100 into the closed position by the user rotating the rotary ring 50 back to the first position without retracting the opening coupling shaft 42.

The process of rotating the rotary ring 50 to its first position is the opposite of the one described above, as detailed in connection with the first embodiment. During rotation of the rotary ring 50, the helical bore 71 of the bearing housing 70 returns the ball 80 to the distal position in the longitudinal groove 51, meanwhile the ball 80 pushes the opening bushing 60 back in the distal direction, by means of the circumferential groove 61, to the first position shown in FIGS. 13a and 14a. By removing the stop 62 and the distal edge 60a′ in the distal direction, the springs 121 and 121′ also push the ball retainers 120 and 120′ back into the closed position. In this way, the closing sections 122 and 122′ are pushed over the flanged bores 112 and 112′ and the balls 113 and 113′ are forced into the flanged bores 112, 112′ and simultaneously into the circumferential grooves 92 formed in the stem 91 of the shaft extension 90. Due to the fact that the width of the edges 93 between the grooves 92 is less than 0.5 mm, preferably less than 0.2 mm, more preferably less than 0.1 mm, the balls 113 and 113′ run off the narrow edges 93 thereby automatically adjusting the shaft extension 90 along the longitudinal axis t to a position wherein the balls 113 and the balls 113′ are seated in a corresponding bore 92. When the ball retainers 120 and 120′ are in the closed position the balls 113 and 113′ prevent further displacement of the shaft extension 90 along the longitudinal axis t. The proximal end of the shaft body 110 is driven by a motor not shown in the figure, and the rotational movement of the shaft body 110 is transmitted to the shaft extension 90 by the pins 118 which extend into the longitudinal grooves 94.

It is noted that the proximal end of the shaft body 110 is not necessarily directly connected to the motor providing the drive force. For example, according to a possible embodiment the axis of the motor is different from the longitudinal axis t of the shaft body 110, for example because the handle contains a handle end bent at an angle. In such a case, the drive is transferred from the motor shaft to the shaft body 110 by means of a bevel gear as known.

Various modifications to the above disclosed embodiments will be apparent to a person skilled in the art without departing from the scope of protection determined by the attached claims.

1. A surgical handpiece (100, 100′) comprising a housing (102) and a drive shaft (10, 10′) arranged therein, characterised in that a stem-engaging bore (11) is defined in the drive shaft (10, 10′), said bore being open at a distal end (10a) of the drive shaft (10, 10′) and having a common longitudinal axis (t) with the drive shaft (10, 10′), at least three first flanged bores (12) defined a wall of the drive shaft (10, 10′) along circumference thereof and perpendicular to the longitudinal axis (t), said first flanged bores opening into the stem-engaging bore (11), flanges (12a) of the first flanged bores being on an inner side of the wall of the drive shaft (10, 10′) facing the stem-engaging bore (11), a fixing ball (13) fitted in each of said first flanged bores (12), and a ball retainer (20, 20′) provided at an outer side of the wall of the drive shaft (10, 10′) and said ball retainer (20, 20′) being movable to a closed position and to an open position and arranged to force the fixing balls (13) into the first flanged bores (12) when in the closed position and to allow a partial exit of the fixing balls (13) from the first flanged bores (12) when in the open position.

2. The surgical handpiece according to claim 1, characterized in that in the closed position of the ball retainer (20, 20′) adjacent fixing balls (13) are spaced at a distance of less than 0.5 mm.

3. The surgical handpiece according to claim 1, characterised in that the ball retainer (20, 20′) has a closing portion (22, 22′) and an opening portion (23, 23′) and is displaceable along the drive shaft (10, 10′), from the closed position to the open position, wherein in the closed position the closing portion (22, 22′) of the ball retainer (20, 20′) is adjacent to the flanged bore (12) and in the open position the opening portion (23, 23′) of the ball retainer (20, 20′) is adjacent to the flanged bore (12)

4. The surgical handpiece according to claim 1, characterized in that the housing (102) includes a handle (40) and a rotary ring (50) rotatable relative thereto about the longitudinal axis (t) between first and second positions, the housing (102) defining an opening bushing (60) and a bearing housing (70) arranged concentrically about the drive shaft (10) and fixed against rotation, an inner side of a wall of the rotary ring (50) having at least one longitudinal groove (51) parallel with the longitudinal axis (t), the opening bushing (60) having an external circumferential groove (61) perpendicular to the longitudinal axis (t), and the bearing housing (70) having at least one external helical bore (71), the rotary ring (50), the opening bushing (60) and the bearing housing (70) being connected by at least one positioning ball (80) seated in the helical bore (71) of the bearing housing (70), the longitudinal groove (51) of the rotary ring (50), and the circumferential groove (61) of the opening bushing (60), so that in the first position of the rotary ring (50), the opening bushing (60) is in a first position along the longitudinal axis (t), in the second position of the rotary ring (50) the opening bushing (60) is in a second position offset from the first position along the longitudinal axis (t), and the opening bushing (60) having a stop (62) cooperating with the ball retainer (20), which, in the first position of the opening bushing (60) is arranged to allow the ball retainer (20) to be in its closed position, and in the second position of the opening bushing (60) is arranged to force the ball retainer (20) into its open position.

5. The surgical handpiece according to claim 4, characterized in that the ball retainer (20) is non-rotatably coupled to the drive shaft (10), and the ball retainer (20) has a profile cooperating with the stop (62), the profile being coupled to the stop (62) in the second position of the opening bushing (60) in a manner blocking rotation of the ball retainer (20) and the drive shaft (10).

6. The surgical handpiece according to claim 1, characterized in that the ball retainer (20) is configured as a sleeve that surrounds the drive shaft (10) and is slidable along the drive shaft, the closing portion (22, 22′) of said ball retainer being a portion of the sleeve having a first inner diameter and the opening portion (23, 23′) of said ball retainer being a portion of the sleeve having a second inner diameter, and wherein the second inner diameter is greater than the first inner diameter.

7. The surgical handpiece according to claim 1, characterized in that the drive shaft (10′) comprises a proximal shaft body (110) and a distal shaft extension (90), the stem-engaging bore (11) opening from a distal end (90a) of the shaft extension (90), and a proximal stem (91) of the shaft extension (90) is engaged in a second stem-engaging bore (111) opening from a distal end (110a) of the proximal shaft body (110).

8. The surgical handpiece according to claim 7, characterized in that at least three second flanged bores (112) are defined in a wall of the drive shaft (110) spaced from said first flanged bores and along circumference of the drive shaft perpendicular to the longitudinal axis (t), opening into the second stem-engaging bore (111), flanges of the second flanged bores being on inner side of a wall of the drive shaft (110) facing the second stem-engaging bore (111), a second fixing ball (113) is fitted in each of the second flanged bores (112), and at an outer side of the wall of the drive shaft (110) a second ball retainer (120) is provided which is movable between closed and open positions, said second ball retainer (120) being arranged to urge the second fixing balls (113) into the second flanged bores (112) in the closed position and to allow partial exit of the second fixing balls (113) from the second flanged bore (112) in the open position, and the proximal stem (91) of the shaft extension (90) comprises a plurality of circumferential grooves (92) extending parallel to each other and formed along circumference of the shaft extension (90) and perpendicular to the longitudinal axis (t), adapted to partially receive the second fixing balls (113), and wherein the shaft body (110) and the shaft extension (90) comprise interconnected elements preventing relative rotation thereof.

9. The surgical handpiece according to claim 8, characterized in that the interconnected elements are a groove (94) formed in the shaft extension (90) parallel with the longitudinal axis (t) and a pin (118) extending through the shaft body (110) and adapted to fit into the groove (94).

10. The surgical handpiece according to claim 8, characterized in that the housing (102) includes handle (40) and a rotary ring (50) rotatable relative thereto between first and second positions about the longitudinal axis (t), an opening bushing (60) inside the housing (102), and a bearing housing (70) arranged concentrically about the drive shaft (10′) and fixed against rotation, wherein an a inner side of a wall of the rotary ring (50) has at least one longitudinal groove (51) parallel with the longitudinal axis (t), an outer side of a wall of the opening bushing (60) has a circumferential groove (61) perpendicular to the longitudinal axis (t), and the bearing housing (70) having at least one external helical bore (71), the rotary ring (50), the opening bushing (60) and the bearing housing (70) being are connected by at least one positioning ball (80) seated in the helical bore (71) of the bearing housing (70), the longitudinal groove (51) of the rotary ring (50), and the circumferential groove (61) of the opening bushing (60), so that in the first position of the rotary ring (50), the opening bushing (60) is in a first position along the longitudinal axis (t), in the second position of the rotary ring (50) the opening bushing (60) is in a second position offset from the first position along the longitudinal axis (t), and the opening bushing (60) having a stop (62) cooperating with the second ball retainer (120), which in the first position of the opening bushing (60) is arranged to allow the second ball retainer (120) to be in its closed position, and in the second position of the opening bushing (60), is arranged to force the second ball retainer (120) into its open position.

11. The surgical handpiece according to claim 10, characterized in that the second ball retainer (120) is non-rotatably coupled to the shaft body (110), and the second ball retainer (120) has a profile cooperating with the stop (62), the profile being coupled to the stop (62) in the second position of the opening bushing (60) in a manner blocking rotation of the second ball retainer (120) and the shaft body (110).

12. The surgical handpiece according to claim 10, characterized in that a second spring (64) is arranged around the drive shaft (10, 10′) forcing the opening bushing (60) into its first position.

13. The surgical handpiece according to claim 10, characterized in that the rotary ring (50) has a first bore (53) and a second bore (54) at its end adjacent the handle (40), the handle (40) has an opening coupling shaft (42) at an end of the handle (40) adjacent the rotary ring (50) adapted to be moved against a third spring (41), a distal end (42a) of the opening coupling shaft (42) is forced into the first bore (53) in the first position of the rotary ring (50) and into the second bore (54) in the second position of the rotary ring (50) by the third spring (41).

14. The surgical handpiece according to claim 13, characterized in that the distal end (42a) of the opening coupling shaft (42) is rounded, and a depth of the first bore (53) is chosen such that when the opening coupling shaft (42) is forced into the first bore (53) the opening coupling shaft (42) is adapted to lock the rotary ring (50) against rotation, and depth of the second bore (54) is sufficient for the rounded distal end (42a) to exit the second bore in response to a torque applied to the rotary ring (50) exceeding a given threshold.

15. The surgical handpiece according to claim 14, characterized in that the distal end (42a) of the opening coupling shaft (42) is rounded and the first bore (53) has a depth of at least 1 mm and the second bore (54) has a maximum depth of 0.2 mm.

16. A rotary tool (200) for a surgical handpiece (100, 100′) according to claim 1, comprising a stem (202) having a longitudinal axis (t′) and a surgical head (204), characterized in that the stem (202) is provided with at least three ball receiving recesses (205) along its circumference perpendicular to the longitudinal axis (t′).

17. The rotary tool according to claim 16, characterized in that adjacent ball receiving recesses (205) are spaced less than 0.5 mm apart.

18. The rotary tool according to claim 16, characterized in that adjacent ball receiving recesses (205) have a radius of curvature equal to the radius of curvature of the fixing balls (13) of the surgical handpiece (100, 100′), and depth of the ball receiving recesses (205) is the same as thickness of a portion of each fixing ball (13) that extends through the flanged bore (12) when the fixing balls (13) are forced by the ball retainer (20) into each flanged bore (12) in the closed position of the ball retainer (20).

19. The surgical handpiece according to claim 1, characterized in that the stem-engaging bore (11) is adapted to receive a stem (202) of a rotary tool (200) such that when the ball retainer (20) is in the closed position, and each fixing ball (13) is forced into each flanged bore (12) by the ball retainer (20), a portion of the fixed ball extends through the flanged bore (12) and fills each ball receiving recess (205) provided in the stem (202) of the rotary tool (200).

20. The surgical handpiece according to claim 1, characterized in that the closed position of the ball retainer (20, 20′) adjacent fixing balls (13) are spaced at a distance of less than 0.2 mm.

21. The surgical handpiece according to claim 4, characterized in that a second spring (64) is arranged around the drive shaft (10, 10′) forcing the opening bushing (60) into its first position.

22. The surgical handpiece according to claim 4, characterized in that the rotary ring (50) has a first bore (53) and a second bore (54) at its end adjacent the handle (40), the handle (40) has an opening coupling shaft (42) at an end of the handle (40) adjacent the rotary ring (50) adapted to be moved against a third spring (41), a distal end (42a) of the opening coupling shaft (42) is forced into the first bore (53) in the first position of the rotary ring (50) and into the second bore (54) in the second position of the rotary ring (50) by the third spring (41).

23. The surgical handpiece according to claim 22, characterized in that the distal end (42a) of the opening coupling shaft (42) is rounded, and a depth of the first bore (53) is chosen such that when the opening coupling shaft (42) is forced into the first bore (53) the opening coupling shaft (42) is adapted to lock the rotary ring (50) against rotation, and depth of the second bore (54) is sufficient for the rounded distal end (42a) to exit the second bore (54) in response to a torque applied to the rotary ring (50) exceeding a given threshold.

24. The surgical handpiece according to claim 23, characterized in that the distal end (42a) of the opening coupling shaft (42) is rounded and the first bore (53) has a depth of at least 1 mm and the second bore (54) has a maximum depth of 0.2 mm.
